(19) United States
(12) Patent Application Publication
(10) Pub. No.: US 2002/0197737 A1
(43) Pub. Date: Dec. 26, 2002

(54) NOVEL METHODS FOR TESTING INHIBITORS OF PAIRED HELICAL FILAMENTS AND USES FOR TREATMENT OF ALZHEIMER'S DISEASE

(76) Inventors: ECKHARD MANDELKOW, BARON-VOGHT-STR.212A (DE); EVA MARIA MANDELKOW, BARON-VOGHT-STR.212A (DE); JACEK BIERNAT, BLOCKHORNER WEIDEN SCHENEFELD (DE); PETER FRIEDHOFF, ELLY-HEUSS-KNAPPWEG 5 GIESSEN (DE)

Correspondence Address: JOSEPH A WILLIAMS JR, MARSHALL O'TOOLE GERSTEIN MURRAY & BORUN, 6300 SEARS TOWER, 233 SOUTH WACKER DRIVE, CHICAGO, IL, 606066402

(21) Appl. No.: 09/319,812

(22) PCT Filed: Dec. 12, 1997
(86) PCT No.: PCT/EP97/07007

(30) Foreign Application Priority Data
Dec. 13, 1996 (EP) 96120120.9

(51) Int. Cl. G01N 33/567; G01N 33/543; A61K 48/00

(52) U.S. Cl. 436/518; 435/7.21; 514/44

(57) ABSTRACT

The present invention relates to the use of inhibitors of intracellular polyanions, for example of RNA or polyanionic polypeptides, or derivatives thereof for the prevention or treatment of Alzheimer disease. In addition, the present invention relates to methods for testing inhibitors for their capacity to inhibit PHF formations and to kits useful in carrying out such methods.

The present invention relates to the use of inhibitors of intracellular polyanions, for example of RNA or polyanionic polypeptides, or derivatives thereof for the prevention or treatment of Alzheimer disease. In addition, the present invention relates to methods for testing inhibitors for their capacity to inhibit PHF formations and to kits useful in carrying out such, methods.

A characteristic feature of brains afflicted with Alzheimer's disease is the abnormal deposition of two types of proteins, the amyloid peptide A beta, and the microtubule-associated protein tau. The latter loses its affinity for the natural partner (microtubules) and instead self-assembles into paired helical filaments (PHFs) which in turn aggregate into neurofibrillary tangles. These filaments have the appearance of two intertwined strands of 10-20 nm diameter, with a repeat distance around 75-80 nm (Wischnik, C. M., et al., (1985) J. Cell Biol. 100, 1905-1912). PHF tau is modified in several ways, most noticeably by phosphorylation, and it is tempting to speculate that the modifications are related to the abnormal aggregation. On the other hand, recombinant tau can aggregate even in an unmodified form when the ionic strength is increased (Wille, H., et al. (1992) J. Cell Biol. 118, 573-584; Crowther, R. A., et al. (1994), FEBS letters 337, 135-138). This tendency is particularly pronounced with tau constructs comprising three of the internal repeats (see Table 1). This agrees well with the observation that the repeat domain constitutes the protease-resistant core of Alzheimer PHFs (Wischik, C. M. et al. (1988) Proc. Natl. Acad. Sci. USA 85, 4506-4510). One explanation is that the repeat domain is capable of forming dimers which in turn promote PHF assembly. This process can be further enhanced by intermolecular disulfide bridges involving Cys 322 in the third repeat (Schweers, O. et al., (1995) Proc. Natl. Acad. Sci. USA 92, 8463-8467; Wile, H. (1992) J. Struct. Biol. 108, 49-61).

On the other hand, tau constructs containing either an additional repeat (no. 2), the domains flanking the repeats, or whole tau isoforms, hardly assemble into PHFs, as if the additional domains acted as inhibitors of the aggregation (Schweers, O. (1995) Proc. Natl. Acad. Sci. USA 92, 8463-846722). This contrasts with the fact that Alzheimer PHFs contain all six tau isoforms of the human CNS (Jakes, R. (1991) EMBO J. 10, 2725-2729), suggesting that the neuron may contain factors that overcome the assembly barrier for full-length tau.

The natural partner of tau, tubulin, associates with tau, polymerizes into microtubules, and thus prevents tau's interaction with itself. Tubulin's C-terminal region, to which tau binds (Littauer, U. Z., et al. (1986) Proc. Natl. Acad. Sci. USA 83, 7162-7166), is unusually acidic, suggesting that tau might respond to other polyanionic molecules. Other prominent polyanions in the cytosol are the various RNA species. These RNA species are, however, also present in normal cells not containing PHFs.

In view of the still outstanding satisfactory explanation what the crucial mechanisms or molecules are that induce tau protein to assemble into PHFs, the technical problem underlying the present invention was to provide further insights into the cellular mechanisms underlying the generation of Alzheimer disease. Such insights should prove useful e.g. in the development of pharmaceutical compositions for preventing or curing Alzheimer disease.

The solution to said technical problem is achieved by providing the embodiments characterized in the claims.

Accordingly, the present invention relates to the use of an inhibitor to an intracellular polyanion or a derivative thereof for the preparation of a pharmaceutical composition for the prevention or treatment of Alzheimer disease.

The term “derivative” as used in connection with the present invention denotes any molecule that is derivable from an intracellular polyanion but retains the capacity to form a complex with the polycationic tau protein or a derivative thereof. Said derivatives may be the product of naturally occurring degradation. Thus, fragments of said intracellular polyanions (or of the tau protein) are specifically included within the term derivative. Derivatives may also be the product of recombinant nucleic acid technology or chemical modification procedures. Fusion products obtainable by recombinant technologies or chemical means which comprise said polyanion or fragment thereof also fall in the term “derivative”.

In spite of the fact that intracellular polyanions naturally occur in normal cells, it was now surprisingly found that said polyanions or derivatives thereof have the capacity to interact with intracellular tau protein and thus induce the formation of PHFs.

In this respect they are similar to polyanions of the extracellular matrix, such as heparin or heparan sulfate, whose effect on PHF assembly has been reported recently (Goedert, M., et al. (1996) Nature 383, 550-553; Perez, M., et al. (1996) J. Neurochem. 67, 1183-1190). While it is conceptually difficult to imagine how components of the extracellular matrix might interact with cytosolic proteins, now that the present invention has been made, the potential role of cytosolic polyanions seems straightforward, making the polyanion-tau connection an attractive model for further investigation of Alzheimer disease.

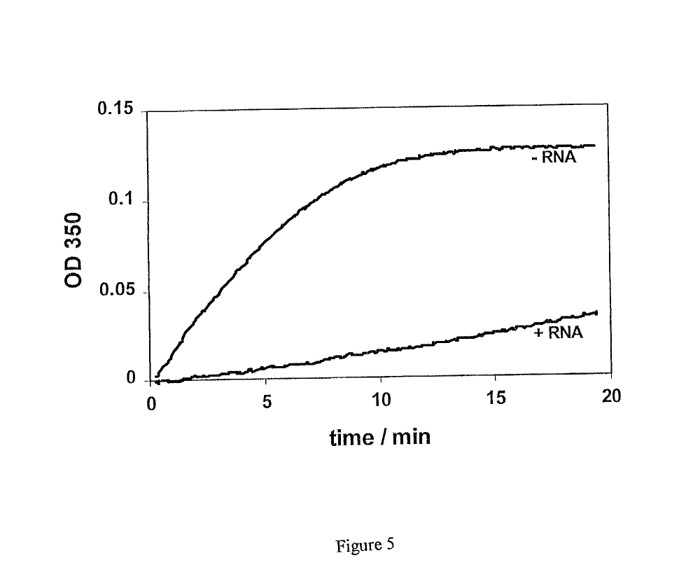

While the applicant is not bound by any scientific theory explaining the invention, the following model would be suitable to account for the data found: Formally speaking, the assembly of tau and tubulin can be described in complementary terms: Tubulin (a polyanion) selfassembles with the help of a polycation (tau), and tau (a polycation) selfassembles with the help of a polyanion (tubulin). The complete microtubule can be described as a heteropolymer: a core filament (poly-tubulin) and an outer coat (poly-tau). In this system, the interaction between tubulin molecules determines the appearance of the structure, while tau seems to be restricted to a helper function. But in principle one could also conceive the reverse situation—a polymeric structure determined by tau, with a tubulin-like molecule in a helper function. This function can be fulfilled by RNA or other intracellular polyanions or derivaties thereof. Indeed, for example, both tubulin and RNA compete for the pool of tau in the cell (Bryan, J. B., et al. (1975) Proc. Natl. Acad. Sci. USA 72, 3570-35743) and in vitro (FIG. 5). Very little is known about the structural requirements of the tubulin-tau interaction, but it is possible that the tau-tau interaction, or the tau conformation, on the surface of a microtubule resembles that in a tau polymer, i.e. in the PHF. This would help to explain why tau selfassembles once the underlying tubulin core is lost (as in Alzheimer's disease). The analogy can be carried one step further: The selfassembly of tubulin can be induced by other polycations, such as DEAE dextran (Erickson, H. P. and Voter, W. A. (1976) Proc. Natl. Acad. Sci. USA 73, 2813-2817), and the selfassembly of tau can be induced by other polyanions such as RNA or heparin. The case of tubulin is instructive because Erickson & Voter (Erickson, H. P. and Voter, W. A. (1976) Proc. Natl. Acad. Sci. USA 73, 2813-2817) showed that the assembly of tubulin by DEAE dextran could be likened to complex coacervation of polyelectrolytes, such that the effective concentration of the anionic protein (tubulin) is increased on the surface of the polycation so that the nucleation barrier is overcome. A similar situation might apply for the assembly of tau; this would be compatible with the different potencies of polyanions to induce PHFs (compare in the examples e.g. the results obtained with tRNA, total RNA, heparin etc.).

The formulation of a pharmaceutical composition comprising said polyanion or derivative thereof is well within the skill of the art. The same holds true for the details of administering said composition. The physician treating the patient will have to take into account, among other parameters, the age, general condition and disease state.

In a preferred embodiment of the use of the present invention, said polyanion is RNA or derived from RNA.

In accordance with the present invention and in line the term “derivative” as defined in connection with the term “polyanion” hereinabove, the term “derived from RNA” denotes any molecule that is obtainable or derivable from naturally occurring intracellular RNA such as naturally or non-naturally occurring degradation products thereof. Since for testing inhibitors in accordance with the present invention for their capacity to prevent or cure Alzheimer disease, recombinantly produced RNAs, chemically altered RNAs or fusion products of RNAs with different molecules may be advantageous, these forms of RNAs are also comprised by the term “derived from RNA”.

In accordance with the present invention, it was surprisingly found that in spite of its abundant presence in normal cells, RNA is capable of promoting the formation of PHFs or PHF-like filaments by tau protein. Whereas the model that in general terms was set forth for polyanions hereinabove may in particular apply to RNA, it is also possible that tau interacts specifically with a particular RNA structure. For example, tau mRNA is transported to the axon hillock in a complex with ribosomes, adaptor and motor proteins so that there is a high local synthesis of tau protein which is destined for slow transport down the axon (Sadot, E., et al. (1995) J. Cell Sci. 108, 2857-2864). The elevated local concentration of RNA and tau protein in the proximal axon could initiate local PHF assembly which would interfere with the axonal transport. This would be compatible with the “dying back” of axons observed in Alzheimer's disease (Braak, E., et al. (1994) Acta Neuropathol. 87, 554-567).

In a particularly preferred embodiment of the use of the present invention, said RNA is nuclear RNA or cytoplasmic RNA.

Whereas the appended examples show that cytoplasmic RNA is suitable to induce PHF formation by cytoplasmic tau and the provision of inhibitors to said RNA is particularly desired, it is to be noted that tau is not exclusively cytosolic: It is known that mRNA transcripts and tau isoforms occur in the nucleus, and particularly in nucleoli (Thurston, V. C., et al. (1996) Chromosoma 105, 20-30; Wang, Y., et al. (1993) J. Cell Biol. 121, 257-267). The role of nuclear tau is not clear, but it does not function as a MAP because there are no nuclear microtubules. Nuclear tau localizes to regions rich in RNA (mostly rRNA and tRNA). Given the results obtained in accordance with the invention one could speculate that nuclear tau and RNA contribute to, or maybe even initiate abnormal assembly of PHFs in Alzheimer neurons. This model is also in line with our results showing that certain tau constructs can translocate into nucleoli after microinjection or transfection of cells. This is particularly apparent for highly basic tau constructs which lack the more acidic N- and C-terminal tails. Since proteolysis of tau is thought to play a role in the initial stages of PHF assembly (Novak, M., et al. (1993) EMBO J. 12, 365-370), one scenario is that truncated tau species migrate from the cytosol to the nucleoli where they aggregate under the influence of RNA.

In a further particularly preferred embodiment of the use of the present invention, said RNA is ribosomal RNA (rRNA), transfer RNA (tRNA) or messenger RNA (mRNA).

In a further preferred embodiment of the use of the present invention, said polyanion is an intracellularly occurring polyanionic protein, a derivative thereof or an intracellularly occurring polyanionic peptide or derivative thereof. The term “derivative” as used here bears the same meaning in connection with proteins as defined hereinabove in connection with polyanions. Thus, said term in particular refers to naturally or non-naturally occurring fragments of said proteins or peptides, to chemically or enzymatically modified (e.g. by phosphorylation) or recombinantly produced proteins or peptides as well as fusion proteins comprising said proteins or peptides or derivatives thereof. Said derivative must also retain the capacity to form a complex with tau protein or a derivative thereof. The polyanionic peptide may be a naturally occurring peptide. It may also be a peptide that is a derivative, in particular a fragment of a polyanionic, polycationic or a neutral protein.

In a preferred embodiment said polyanionic polynucleotide, peptide or derivative thereof is poly-glu. In another preferred embodiment said polyanionic polypeptide, peptide or derivative thereof is derived from tubulin. It may comprise for example a partial sequence of α- or β-tubulin, an isoform thereof, or a posttranslational modification. One of such modifications may be, for example, poly-glutamylation.

In a particularly preferred embodiment, said polyanionic peptide comprises the C-terminal region of tubulin. The C-terminal region of tubulin is known to contain various glutamic acid residues and shows strong polyanionic properties. It is, as has been outlined above, also the part of tubulin that under normal physiological conditions intracellularly binds to tau. It is therefore envisaged by the present invention that a degradation product of tubulin consisting of or comprising the C-terminal region plays a role in the induction of Alzheimer disease. Naturally, the peptide comprising the C-terminal region of tubulin may be a derivative of tubulin with the features of a derivative as has been defined hereinabove.

Said peptide may be used as a potential nucleation germ for the formation of Alzheimer PHF's. Said PHF formation can be assayed within cells or in in vitro assays. Said β-tubulin derived peptide may have, for example, the following amino acid sequences:

(a) EEEEGEDEA,

(b) GEFEEEEGEDEA, or

(c) GEFEEEEGEDEA;

|

En

wherein peptide (c) is polyglutamylated at one glutamic acid residue with n=0, 1, 2, 3, 4, 5, 6, 7 or 8 further glutamic acid residues.

The polyanionic proteins, peptides or derivatives thereof may be found in the cytosol or in the nucleus.

An additional preferred embodiment of the present invention relates to a use as defined hereinabove, wherein said inhibitor is a polycation or a ribozyme.

The invention also relates to a kit comprising

(a) recombinantly produced tau protein or a derivative thereof; and

(b) a polyanion.

The term “derivative” of tau protein is intended to mean throughout this specification any recombinantly produced protein that has all or part of the structural and/or biological features of tau protein but being distinct therefrom. Examples of such derivatives are proteins that are devoid of the N- or C-terminal tail of tau protein (see, e.g., Table 1). “Derivative” is also intended to mean chemically or enzymatically modified (e.g. phosphorylated) tau proteins or fusion proteins comprising all or part of tau. “Tau protein” as used throughout the specification is intended to mean any of the isoforms of tau protein, preferably of human tau protein.

The kit of the invention is useful for testing inhibitors to the formation of PHFs. As will be discussed in more detail in connection with the method of the invention, the compounds of the kit of the invention can be mixed with a prospective inhibitor under suitable conditions. Inhibition of PHF formation would identify the prospective inhibitor as a candidate for further development of a pharmaceutical composition.

Preferably, the polyanion in the kit of the invention is RNA or a polyanionic protein, peptide or derivative thereof. If said polyanion is RNA, it is most preferably tRNA or rRNA.

Additionally, the present invention relates to an in vitro method for testing an inhibitor of paired helical filament (PHF) formation of PHF-like formation comprising

(a) combining in a test vial tau protein or a derivative thereof with a polyanion and the prospective inhibitor; and

(b) testing whether the presence of the prospective inhibitor reduces or inhibits PHF formation.

With the method of the present invention it has become feasible to identify inhibitors to polyanions that are expected to also have a direct or indirect impact on PHF formation or PHF-like formation when administered to humans. The person skilled in the art is capable of selecting a suitable test vial for carrying out the method of the invention. The compounds included for the test in the vial may be added all at the same time or successively. It is thus envisaged that the tau protein or derivative thereof is first incubated with an intracellularly occurring polyanion and the prospective inhibitor is added after a certain preincubation time. Thus, it is not only possible to detect prevention of PHF formation or PHF-like formation but also to investigate total or partial reversal of PHF or PHF-like formation. With the method of the invention it is also possible to determine any inhibitor that prevents or reverses PHF or PHF-like filament formation after an induction or onset of said formation by polyanions as defined hereinabove.

In a more preferred embodiment said polyanion in said method is RNA or poly-glu, or a peptide derived from tubulin. If said polyanion is RNA, it is most preferably tRNA or rRNA. If said polyanion is a peptide derived from tubulin, it comprises most preferably the C-terminal region of tubulin or a derivative thereof.

As has been shown in accordance with the present invention, it is possible to induce PHF formation in vitro with poly-glu. Poly-glu as used in the present invention can be a mixture of poly-glu-molecules of different length, depending on its specification and manufacturer.

The polyglutamines may be a mixture of polyglutamines with varying lengths. In a particularly preferred embodiment said poly-glu comprises 8 to 12 glutamic acid residues. An example of such a poly-glu is poly-glu 1000 from Sigma, which has an average length of 8 amino acids. Commercially available mixtures of polyglutamic acid residues are cheap and are useful in routine-investigations of PHF-formation and thus particularly appropriate for the identification of inhibitors of PHF formation.

The person skilled in the art is also capable of selecting or devising suitable readout systems for evaluating the efficacy of the inhibitor. For example, PHF or PHF-like formation may be visualized using light scattering that is detected and evaluated with a spectrophotometer. An example of such an evaluation is provided in the appended examples.

The present invention further relates to a method for testing the onset of Alzheimer disease comprising

(a) overexpressing tau protein or a derivative thereof in a cell or introducing tau protein or a derivative into a cell; and

(b) testing a prospective inhibitor for the inhibition or reduction of PHF or PHF-like filament formation.

The cell employed in the method of the invention may be, for example, a neuronal cell, a neuroblastoma cell or a cell obtained from the hippocampus. Methods of overexpressing proteins in cells and for introducing a protein into a cell are well known in the art. Again, devising a suitable readout system for testing the inhibition is also within the skills of the person skilled in the art. The present method of the invention in all its embodiments described in the specification also envisages that the inhibitor is expressed or overexpressed in said cell or introduced into said cell.

Preferably, the method of the invention further comprises

(a″) introducing a polyanion susceptible or capable of inducing PHF- or PHF-like filament formation into said cell or overexpressing said polyanion in said cell.

The polyanion as defined hereinabove may be introduced into said cell by conventional means, for example by microinjection or electroporation. The polyanion can be introduced prior to, after, or at the same time with the prospective inhibitor into the cell. This embodiment of the present invention has the particular advantage that by introducing the polyanion, testing for the inhibitor may be accelerated. Further, once the inhibitor is identified as such, detailed studies on the onset of Alzheimer disease can be carried out, e.g. by titration experiments.

Additionally, the invention relates to a method comprising

(a) introducing tau protein or a derivative thereof in a cell or overexpressing tau protein or a derivative thereof in a cell;

(b) introducing a polyanion into said cell or overexpressing a polyanion in a cell; and

(c) testing for the induction of PHF- or PHF-like formation.

This embodiment of the method of the invention is particularly suitable to study the onset of Alzheimer disease. Further, the role of different intracellular polyanions or derivatives thereof in the onset of Alzheimer diesease may be studied. This role may be a direct or indirect one.

Preferably, the polyanion employed in the method of the invention is RNA, a polyanionic protein or peptide or derivative thereof. Said RNA is most preferably nuclear RNA or cytoplasmic RNA, in particular rRNA, tRNA or mRNA whereas said polyanionic peptide most preferably is poly-glu or an anionic peptide derived from tubulin or comprises the C-terminal region of tubulin or a derivative thereof.

Preferably, in the method of the present invention, the formation of the PHFs is detected by fluorescent staining, for example, with thioflavin. Of course, the fluorescent staining with thioflavin also provides the person skilled in the art with information on the inhibition of PHF formation.

The above described embodiments of the method of the invention may be an in vitro or an in vivo method.

Finally, the present invention relates to methods of preventing or treating Alzheimer disease in humans comprising administering to a patient in need thereof a pharmaceutical composition comprising an inhibitor for PHF formation as described herein above. Conditions and routes of administration have also been defined hereinabove or can be derived by the physician handling the case.

The therapeutically useful compounds identified according to the method of the invention may be administered to a patient by any appropriate method for the particular compound, e.g., orally, intravenously, parenterally, transdermally, transmucosally, or by surgery or implantation (e.g., with the compound being in the form of a solid or semi-solid biologically compatible and resorbable matrix) at or near the site where the effect of the compound is desired. Therapeutic doses are determined to be appropriate by one skilled in the art. Preferably, the dose to be administered is in the range of 1 ng to 10 mg per kg of body weight per day.

These and other embodiments are disclosed and encompassed by the description and examples of the present invention. For example, further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The uses and methods of the invention can be used for the treatment of all kinds of diseases hitherto unknown as being related to or dependent on Alzheimers disease. The methods and uses of the present invention may be desirably employed in humans, although animal treatment is also encompassed by the methods and uses described herein.

Legends to Figures and Tables:

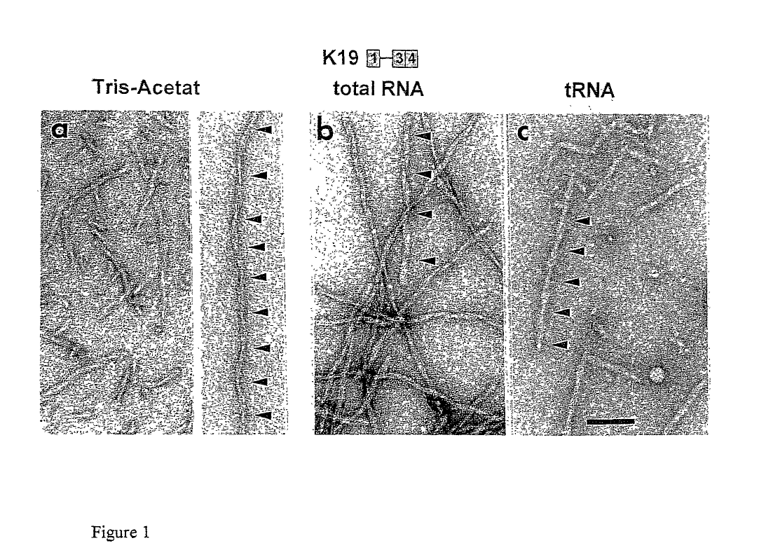

FIG. 1: PHFs assembled from 3-repeat construct K19. (a) 0.2 M TrisHCl, 0.2 M Na acetate; 2 mM K19, 7 weeks, (b) total RNA (0.5 mg/ml), 200 μM K19, 14 h, (c) tRNA (0.5 mg/ml), 200 μM K19; 14 h.

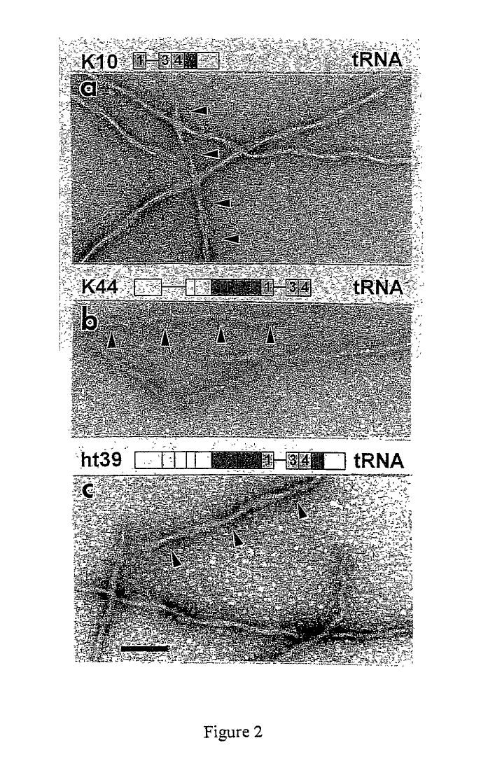

FIG. 2: PHFs assembled from constructs containing 3 repeats and extensions in the presence of 0.5 mg/ml tRNA. (a) 40 μM K10 (3 repeats plus C-terminal tail), (b) 40 μM K44 (3 repeats and N-terminal domain), (c) 40 μM htau39 (the second-largest tau isoform).

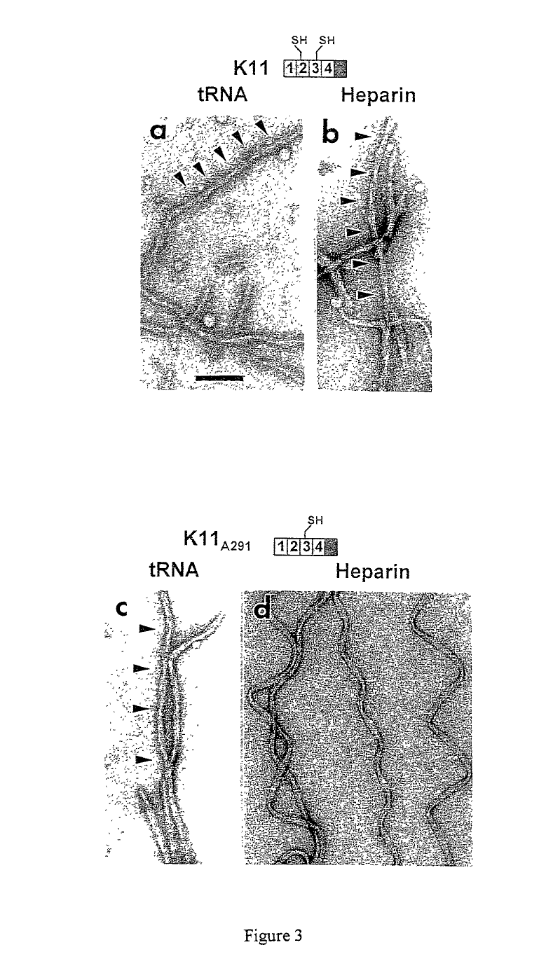

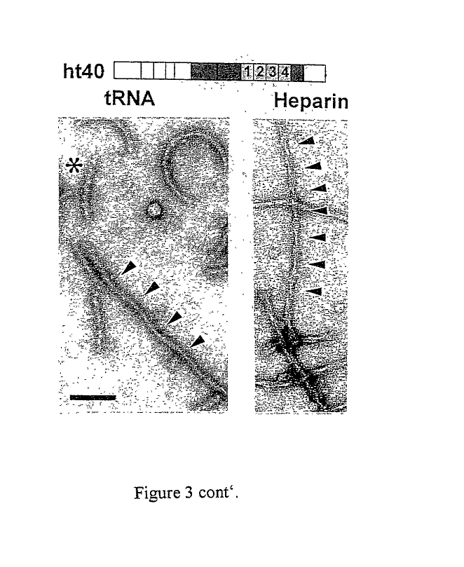

FIG. 3: PHFs assembled from 4 repeat tau constructs or isoforms. K11 (400 μM) with (a) 0.5 mg/ml tRNA and (b) 10 μM heparin, or K11 mutant Cys291Ala (400 μM) with (c) 0.5 mg/ml tRNA and (d) 10 μM heparin. (e) 40 μM htau40 (largest tau isoform) with 0.5 mg/ml tRNA or (f) 40 μM htau40 with 10 μM heparin. Note that full length tau assembles much less readily than the repeat domain, and that the filaments show more polymorphism.

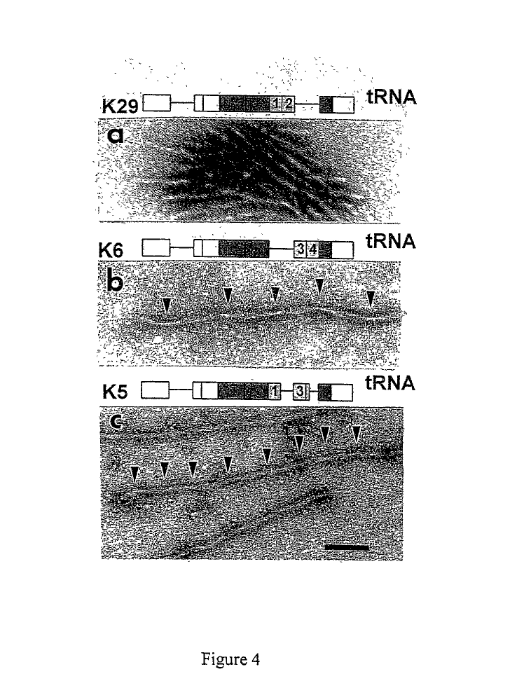

FIG. 4: Assembly experiments of tau constructs with two repeats under the influence of tRNA. (a) K29 600 μM (repeat 1 and 2), (b) K6 200 μM (repeat 3 and 4), (c) K5 400 μM (repeat 1 and 3). Removal of the repeats decreases tau's tendency to form PHFs.

FIG. 5: Assembly of microtubules (10 μM) in the presence of htau40 (2 μM), without or with 0.2 mg/ml total RNA. Note the inhibition of microtubule assembly by RNA which competes with tubulin for tau protein.

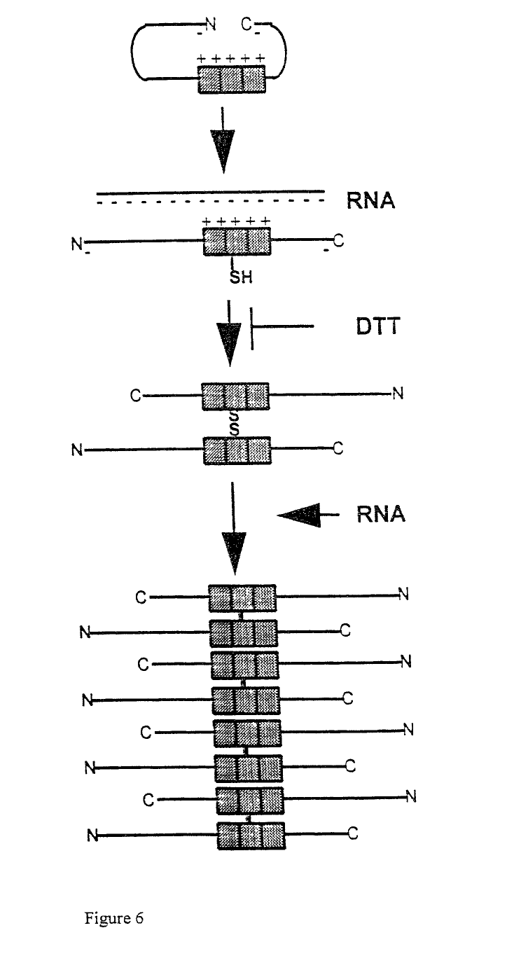

FIG. 6: Model of the influence of RNA or other anions on the assembly of PHFs from tau protein. To form PHFs, tau molecules initially dimerize with their repeat domains (Wille, H. et al., (1992) J. Cell Biol. 118, 573-584). The regions of tau flanking the repeats on either side (particularly the acidic N-terminal and C-terminal tails) are normally folded over the repeats, thus preventing dimerization and subsequent PHF assembly. Polyanions counteract the folded conformation, opening the repeats up to dimerization and PHF assembly.

Table 1: Diagrams of tau isoforms and constructs used in this study and their propensity to form PHF-like filaments in standard buffer with 0.5 mg/ml tRNA.

The examples illustrate the invention.

### EXAMPLE 1

Stimulation of the Assembly of PHFs from tau Protein by the Intracellular Polyanion RNA

Constructs of the tau protein (see Table 1) were designed and expressed in E.coli as described (Biernat, J. et al., (1992) EMBO J. 11, 1593-1597). The numbering of the amino acids is that of the isoform htau40 containing 441 residues (Goedert, M. et al., (1989) EMBO J. 8, 393-399). The proteins were expressed and purified as described elsewhere making use of the heat stability and FPLC Mono S (Pharmacia) chromatography (Gustke, N. et al., (1994) Biochemistry 33, 9511-9522). The purity of the proteins were analyzed by SDS-PAGE.

To assess the effect of different polyanions on the assembly of tau into PHFs we initially chose constructs consisting essentially of the repeats. Assembly of these and others constructs to PHF-like filaments was measured as follows: Varying concentrations of tau isoforms or tau constructs (typically in the range of 40-400 μM) in volumes of 15 to 500 ul were incubated at 37 C. in 100 mM TrisHCl, pH 6.8 containing various anionic cofactors: Total RNA from yeast (Boehringer) or bovine liver (Sigma), tRNA from bovine liver (Sigma), rRNA from bovine liver (Sigma), or heparin (Sigma) were varied between 0.05 and 0.5 mg/ml. Incubation times varied between 2 hours up to several days. Assembly reactions without polyanions were carried out as described in Schweers, O. et al., (1995) Proc. Natl. Acad. Sci. USA 92, 8463-8467. Assembly was monitored via electron microscopy for which the protein solutions were placed on 600-mesh carbon-coated copper grids and negatively stained with 2% uranyl acetate. The specimen were examined in a Philips CM12 electron microscope at 100 kV.

Construct K12 (=3 repeats plus a C-terminal extension, Tab. 1) was shown earlier to have a high tendency to form PHFs because it readily forms dimers and higher aggregates (Wille, H. et al., (1992) J. Cell Biol. 118, 573-584), and it contains only one cysteine (Cys 322) which favors the formation of intermolecular disulfide bridges (Schweers, O. et al., (1995) Proc. Natl. Acad. Sci. USA 92, 8463-8467). Construct K19 (=3 repeats only) assembles even better (FIG. 1). The assembly can be driven by increasing the buffer concentration (e.g. up to 0.4 M TrisHCl pH 6.8, 0.4 M Na-acetate); the effect becomes particularly evident by allowing the buffer to evaporate slowly and thereby increasing the protein and salt concentrations. The filaments have the typical PHF-like appearance, with widths varying between 10 and 20 nm, and a cross-over repeat of about 75 nm.

Several RNA species (total RNA from yeast or bovine liver, rRNA, tRNA, or poly(A)) were able to promote the assembly of K19 noticeably, reducing the assembly times from days to hours, and again forming the typical PHF-like structures (FIG. 1). The effects are analogous to those described for polyanions of the extracellular matrix such as heparin (FIG. 1; Perez, M. et al., (1996) J. Neurochem. 67, 1183-1190, Goedert, M. et al., (1996) Nature 383, 550-553).

The next question addressed was which tau isoforms or constructs would assemble under the influence of RNA. As noticed earlier, it is difficult to obtain PHFs from constructs containing domains outside the repeats or the second repeat, at least in conditions where assembly is driven by high ionic strength and high protein concentration (Crowther, R. A. et al., (1994) FEBS Lett. 337, 135-138; Wille, H. et al., (1992) J. Cell Biol. 118, 573-584). This would suggest that some non-repeat domains of tau prevent PHF formation. The same tendency is observed here again, but now it is found that the inhibition can be easily overcome by RNA in most cases. FIG. 2 shows examples of PHFs made from the construct K10 where the entire C-terminal tail has been added to the 3-repeat domain. This construct slowly develops PHFs when incubated in high salt (0.4 M TrisHCl, pH 6.8, 0.4 M Na-acetate) and at high protein concentration (about 1 mM) over the course of several days. By contrast, with tRNA (0.5 mg/ml) one obtains filaments rapidly, within hours, (FIG. 2a). The next step was to take 3 repeats plus the N-terminal domain (construct K44); this also readily forms filaments in the presence of tRNA (FIG. 2b). Finally the full-length three repeat isoforms htau39 was tested. In high salt, this isoform is hardly capable of forming PHFs, but with tRNA one obtains PHFs in hours even at low protein concentration (FIG. 2c). Note however that the aggregation of htau39 is not as fast and efficient as with the 3-repeat construct alone (K19). In our earlier studies we had found that 4-repeat isoforms are inefficient in PHF assembly because the two cysteines can form intra-molecular disulfide bonds which stabilize the “compact” monomer and prevent dimerization. Since dimers are building blocks of PHFs, the extra repeat (no. 2) effectively acted as a PHF inhibitor (Schweers, O. et al., (1995) Proc. Natl. Acad. Sci. USA 92, 8463-8467). However, this inhibition can be overcome by tRNA. The four-repeat domain K11 forms filaments, part of which have the authentic twisted appearance while others are straight (FIG. 3a). If Cys291 in the second repeat is mutated into Ala (leaving only the single Cys322 in the third repeat) the assembly of PHFs becomes highly efficient again (FIG. 3c,d). Some of these filaments show a supercoil of diameter 40-100 nm and pitch 150-200 nm. Extending K11 in the N- and C-terminal direction is equivalent to the largest tau isoform htau40. In this case, even with tRNA it is difficult to obtain bona fide PHFs. Instead one observes a mixture of polymorphic filaments, including thin straight filaments, twisted filaments, and “spiny” filaments with protrusions at ˜20 nm intervals (FIG. 3e, f).

Since repeats were considered important for PHF assembly it was next investigated how the removal of repeats would affect the process. Several constructs derived from the isoform htau40 in which the number of repeats was reduced to 3, 2, 1 or 0 we have made. The loss of repeats lead to a reduction in PHF assembly, even in the presence of tRNA. Constructs with two repeats were less efficient while constructs with only one repeat (K13, K14, K15) or no repeat (K23) did not form any PHF-like filaments (FIG. 4a-c, Table 1).

The comparison of different tau domains shows that RNA-induced assembly of PHFs works best with the 3-repeat construct K19, while adding domains outside the repeats or the repeat no. 2 have an inhibitory effect which must be counteracted by higher protein concentration and longer incubation times. The data can be summarized by the model of FIG. 6 which is an extension of the dimerization model proposed earlier. The domain consisting of repeats 1, 3, and 4 dimerize most easily on account of the single Cys 322 in repeat no. 3 which can enter inter-molecular disulfide bonds. The resulting antiparallel dimers have a high tendency to interact with others to form PHFs, and the process can be inhibited by reducing agents such as DTT (see also Example 2). The repeat no. 2 is inhibitory because its extra Cys 291 can form an intra-molecular disulfide bond, making the molecule compact (as judged from its migration on native gels) and unable to dimerize. In practice, whether or not a disulfide bond is intra- or intermolecular will depend on the protein concentration, the molecular collision frequencies, the rate of oxidation and other parameters. This would explain why even 4 repeat domains can form dimers at higher concentrations, albeit less readily.

The inhibitory effect of the N- and C-terminal tails could be explained by their conformation. Both tails are acidic and therefore could fold back onto the repeat domain. Such an interaction would be consistent with the flexible nature of the polypeptide chain (Schweers, O. et al., (1994) J. Biol. Chem. 269, 24290-24297), with the reactivity of certain antibodies (Lichtenberg-Kraag, B. et al., (1992) Proc. Natl. Acad. Sci. USA 89, 5384-5388), and with electron microscopic or fluorescence energy transfer experiments (Schweers O. et al., (1995) Proc. Natl. Acad. Sci. USA 92, 8463-8467, Wille, H. et al., (1992) J. Cell Biol. 118, 573-584). In the model it is assumed that the folded-back tails somehow protect the repeat domain, making it unavailable for dimerization and PHF assembly. However, the folded state can be “forced open” by polyanions such as RNA. If this happens, dimerization is possible again, and once the dimers are stabilized by intermolecular disulfide bonds they can be stably incorporated into PHFs. This would explain why larger tau constructs assemble less efficiently in the absence of polyanions, and that their assembly is prevented by DTT, pointing to the role of disulfides. In this model, the “open” conformation of tau can interact with other tau molecules. In addition, it is possible that the same conformation is also the one that interacts with different polyanions, particularly microtubules. Thus, the open conformation could be viewed as the physiologically active one, while the folded conformation would represent an inactive storage form.

### EXAMPLE 2

Structure and Kinetics of PHF Assembly

Perhaps the most remarkable feature of PHF assembly from different tau constructs is the similarity in the resulting structure. The majority of the fibers have the appearance of two strands twisted around one another, with widths of 10-20 nm and cross-over repeats on the order of 75 nm. (However there was also a population of filaments with cross over periodicity of about 120 nm (100-130)). The type and composition of the constructs, or the agent promoting the assembly, seem to matter only in a second approximation. The simplest interpretation is that PHFs are built on a common principle. The smallest construct from which PHFs was obtained is the construct K19 (3 repeats), and therefore it is likely that PHF assembly is based on the interactions between at least one (probably several) of the repeats. Although the PHF preparations are dominated by twisted fibers there is usually a fraction which appear straight, but of comparable width (20 nm). Similar straight filaments have been observed in other assembly conditions of tau (e.g. de Ancos, J. G. et al., (1993) J. Biol. Chem. 268, 7976-7982, Lichtenberg-Kraag, B. and Mandelkow, E. M. (1990) J. Struct. Biol. 105, 46-53, Wilson, D. M. and Binder, L. I. (1995) J. Biol. Chem. 270, 24306-24314), and even in Alzheimer PHFs (Crowther, R. A. (1991) Proc. Natl. Acad. Sci. USA 88, 2288-2292). We did not observe a defined influence of tau domains on the straight or twisted fraction of filaments. In this regard the results presented here differ somewhat from those reported earlier with heparin. Goedert et al. (Goedert, M. et al., (1996) Nature 383, 550-553) observed only straight filaments with 4-repeat isoforms while we find straight and twisted filaments (FIG. 3d, arrow). Perez et al. ((1996) J. Neurochem. 67, 1183-1190) reported mostly untwisted filaments with both 3- and 4-repeat tau constructs. Since a straight filament may gradually convert into a twisted one, and vice versa, it is likely that the two appearances are closely related (as noted by Crowther, R. A. (1991) Proc. Natl. Acad. Sci. USA 88, 2288-2292).

As shown before (see Example 1), the rate of PHF assembly is enhanced if tau monomers are first allowed to form dimers stabilized by intermolecular disulfide bonds involving Cys 322. The influence of disulfide bridge formation on the RNA-induced assembly of PHFs was therefore investigated. Indeed, when disulfide bridges were prevented by reducing agents (such as DTT), fiber formation was strongly reduced. The same effect was achieved by mutating Cys into Ala. These observations are in agreement with the assembly model proposed earlier based on disulfide-crosslinked tau dimers (Schweers, O. et al., (1995) Proc. Natl. Sci. USA 92, 8463-8467). On the other hand, this model also postulated that only constructs with one cysteine would form dimers (and thus PHFs), while others with two cysteines (Cys 291 and 322 in repeats 2 and 3) would form intra-molecular disulfide bridges, leading to a “compact” formation of tau which would not contribute to PHF assembly. The present data show that even 4-repeat tau (with 2 cysteines) can assemble into PHFs in the presence of RNA, albeit with low efficiency (FIG. 3). The simplest explanation is that RNA prevents the compact conformation leading to intramolecular bonds, at least for part of the molecules, so that inter-molecular dimerization is possible. Consistent with this interpretation, the mutant Cys291-Ala shows abundant PHF assembly since the lone Cys 322 can only enter inter-molecular disulfide bonds (FIG. 3c, d).

### EXAMPLE 3

Microtubule Assembly Assay

The role of RNA as a “scavenger” of tau can be demonstrated most directly by a microtubule assembly assay. In the experiment of FIG. 5 (upper curve), microtubule assembly was monitored by light scattering in a Kontron UVIKON 810 spectrophotometer by absorption at 350 nm. 10 μM tubulin dimers (purified as described in (Mandelkow, E. M. et al., (1985) J. Mol. Biol. 185, 311-327) were incubated in 80 mM PIPES, 1 mM EGTA, 1 mM MgCl2, 1 mM DTT, 1 mM GTP, pH 6.8 with or without the addition of 0.2 mg/ml RNA in a 10 mm cuvette. Polymerization was started at 37° C. by adding a small volume of tau to a final concentration of 2 μM. The concentration of tubulin (10 μM) was chosen such that it would not self-assemble but required tau for nucleation and stabilization. However, when RNA was added as well, tau was competed away so that microtubule assembly was inhibited (lower curve).

### EXAMPLE 4

Monitoring PHF Assembly or Inhibition by Thioflavin Fluorescence

Thioflavin is a fluorescent dye that is commonly used to stain brain sections to detect the presence of Alzheimer neurofibrillary tangles. Its fluorescence changes when it binds to paired helical filaments (PHFs), the fibrils that make up the neurofibrillary tangles in Alzheimer brain tissue (see e.g. LeVine, H. (1993). Protein Science 2, 404-410). We have recently found that the thioflavin fluorescence can be used as a quick method to monitor the assembly of tau or tau derivatives into PHFs in vitro (Friedhoff et al., manuscript in preparation). This method is useful in determining the assembly capacity of different tau constructs, the capacity of various polyanionic substances to promote PHF assembly (e.g. RNA, poly-glu, or tubulin peptides), and the effect of potential drugs to inhibit PHF assembly. The proteins can be prepared similar to Example 3, placed into the cuvette of a standard spectrofluorimeter in the presence of thioflavin, and PHF assembly is monitored using an excitation wavelength of 440 nm and observing the fluorescence at an emission wavelength of 480-500 nm. The same principle could be applied to 96 well plates and fluorescence detection which would be useful for large scale screening of inhibitors of PHF assembly.

1. Use of an inhibitor to an intracellular polyanion or a derivative thereof for the preparation of a pharmaceutical composition for the prevention and/or treatment of Alzheimer disease.

2. The use according to claim 1 wherein said polyanion is RNA or derived from RNA.

3. The use according to claim 2 wherein said RNA is nuclear RNA or cytoplasmic RNA.

4. The use according to claim 2 or 3 wherein said RNA is ribosomal RNA (rRNA), transfer RNA (tRNA) or messenger RNA (mRNA).

5. The use according to claim 1 wherein said polyanion is a intraceillulariy occurring polyanicnic polypeptide or peptide or a derivative thereof.

6. The use according to claim 1 wherein said polyanion is poly-glu.

7. The use according to claim 5 wherein said peptide is an anionic peptide derived from tubulin.

8. The use according to claim 7 wherein said peptide comprises the C-terminal region of tubulin.

9. The use according to any one of claims 1 to 4 wherein said inhibitor is a polycation or a ribozyme.

10. Kit comprising
(a) recombinantly produced tau protein or a derivative thereof; and
(b) a polyanion.

11. The kit according to claim 10 wherein said polyanion is RNA or a polyanionic protein, peptide or derivative thereof.

12. The kit according lo claim 11 wherein said RNA is tRNA or rRNA.

13. An in vitro method for testing an inhibitor of paired helical filament (PHF) formation or PHF-like formation comprising
(a) combining in a test vial tau protein or a derivative thereof with a polyanion and the prospective inhibitor; and
(b) testing whether the presence of the prospective inhibitor reduces or inhibits PHF formation.

14. The method of claim 13, wherein said polyanion is RNA as defined in any one of claims 2 to 4, or poly-glu, or a peptide derived from tubulin as defined in claim 7 or 8.

15. A method for testing an inhibitor for the inhibition or reduction of PHF- or PHF-like filament formation comprising
(a) overexpressing tau protein or a derivative thereof in a cell or introducing tau protein or a derivative thereof into a cell; and
(b) testing the prospective inhibitor of PHF- or PHF-like filament formation.

16. The method according to claim 15 further comprising
(a′) introducing a polyanion susceptible or capable of inducing PHF- or PHF-like filament formation into said cell or overexpressing said polyanion in said cell.

17. A method comprising
(a) introducing tau protein or a derivative thereof into a cell or overexpressing tau protein or a derivative in a cell;
(b) introducing a polyanion into said cell or overexpressing a polyanion in a cell, and
(c) testing for the induction of PHF- or PHF-like filament formation.

18. The method according to any one of claims 14 to 17 wherein said polyanion is RNA or a polyanionic protein or peptide or a derivative thereof.

19. The method according to claim 18 wherein said RNA is nuclear RNA or cytoplasmic RNA and said peptide is poly-glu, or is derived from tubulin or comprises the C-terminal region of tubulin, or a derivative thereof.

20. The method according to claim 18 or 19 wherein said RNA is rRNA, tRNA or mRNA.

21. The method of any one of claims 13 to 20, wherein the formation of PHFs is detected by fluorescent staining with thioflavin.

22. A method of treating or preventing Alzheimer disease in humans comprising administering to a patient in need thereof a pharmaceutical composition comprising an inhibitor for PHF formation as defined in any one of claims 1 to 21.
